(19) United States
(12) Patent Application Publication
(10) Pub. No.: US 2019/0314036 A1
(43) Pub. Date: Oct. 17, 2019

(54) Compression Clip Structure

(71) Applicant: Shu-Han Huang, Changhua County (TW)

(72) Inventor: Shu-Han Huang, Changhua County (TW)

(21) Appl. No.: 15/950,245

(22) Filed: Apr. 11, 2018

(51) Int. Cl. A61B 17/132; F16B 2/10; F16B 2/08

(52) U.S. Cl. CPC A61B 17/1322; F16B 2/08; F16B 2/10

(57) ABSTRACT

A compression clip structure includes a first clip, a second clip, a shaft, an elastic member and a fastener. The first clip has a first clamping portion, a mounting portion and a first pivot portion. The second clip has a second clamping portion, a push portion and a second pivot portion. The shaft extends through the first pivot portion and the second pivot portion. The elastic member is mounted on the shaft and provided with two resting portions resting on the first clip and the second clip. The fastener extends through a fitting hole of the push portion and has a pressing portion and a control portion. Thus, the compression clip structure clamps a compression bandage, and the fastener secures the compression clip structure and the compression bandage, so as to stop the bleeding stably.

### BACKGROUND OF THE INVENTION

### 1. Field of the Invention

The present invention relates to a clip and, more particularly, to a compression clip structure for clamping a compression bandage or a tourniquet during a stop bleeding process.

### 2. Description of the Related Art

A compression bandage or tourniquet is used to stop the flow of blood from the artery by compression when a person is wounded and has a larger cut. The compression bandage is located at a position between the heart and the wounded part of the human body. The compression bandage is directly bound on the human body or secured by a securing mechanism, such as a pen, stick or the like. However, the compression bandage easily becomes loosened due to vibration during movement or transportation, thereby decreasing the stanching function of the compression bandage.

### BRIEF SUMMARY OF THE INVENTION

The primary objective of the present invention is to provide a compression clip structure for clamping a compression bandage during a stop bleeding process to stop the bleeding exactly and to enhance the stanching function.

In accordance with the present invention, there is provided a compression clip structure comprising a first clip, a second clip, a shaft, an elastic member and a fastener. The first clip has a first clamping portion, a mounting portion and a first pivot portion. The first clamping portion of the first clip is provided with an opening and a clamping zone. The mounting portion of the first clip is provided with at least one through hole. The first pivot portion of the first clip is located between the first clamping portion and the mounting portion and has two first ears formed on two sides thereof. Each of the two first ears of the first clip is provided with a first pivot hole. The second clip has a second clamping portion, a push portion and a second pivot portion. The second clamping portion of the second clip is provided with a clamping end. The push portion of the second clip is provided with a fitting hole. The second pivot portion of the second clip is located between the second clamping portion and the push portion and has two second ears formed on two sides thereof. Each of the two second ears of the second clip is provided with a second pivot hole. The shaft extends through the first pivot hole of each of the two first ears and the second pivot hole of each of the two second ears, so that the first clip and the second clip are connected pivotally by the shaft. The elastic member is mounted on the shaft and provided with two resting portions resting on the first clip and the second clip, so that the clamping zone of the first clip and the clamping end of the second clip are directed toward each other, and the first clip and the second clip contain an elastic clamping force. The fastener extends through and is movable in the fitting hole of the second clip. The fastener is provided with a pressing portion and a control portion. The control portion of the fastener controls movement of the pressing portion, so that the pressing portion of the fastener is located at a predetermined position.

According to the primary advantage of the present invention, the compression clip structure is used to clamp the compression bandage, and the fastener is used to secure the compression clip structure and the compression bandage, so as to stop the bleeding stably.

Further benefits and advantages of the present invention will become apparent after a careful reading of the detailed description with appropriate reference to the accompanying drawings.

### BRIEF DESCRIPTION OF THE SEVERAL VIEWS OF THE DRAWING(S)

FIG. 1 is a perspective view of a compression clip structure in accordance with the preferred embodiment of the present invention.

FIG. 2 is an exploded perspective view of the compression clip structure as shown in FIG. 1.

FIG. 3 is a top view of a first clip of the compression clip structure in accordance with the preferred embodiment of the present invention.

FIG. 4 is a bottom view of a second clip of the compression clip structure in accordance with the preferred embodiment of the present invention.

FIG. 5 is a schematic side operational view of the compression clip structure in accordance with the preferred embodiment of the present invention.

FIG. 6 is a schematic operational view of the compression clip structure as shown in FIG. 5.

FIG. 7 is a schematic side operational view of the compression clip structure in accordance with another preferred embodiment of the present invention.

FIG. 8 is a schematic operational view of the compression clip structure as shown in FIG. 7.

FIG. 9 is a perspective operational view of the compression clip structure in accordance with the preferred embodiment of the present invention.

FIG. 10 is a schematic top operational view of the compression clip structure in accordance with the preferred embodiment of the present invention.

### DETAILED DESCRIPTION OF THE INVENTION

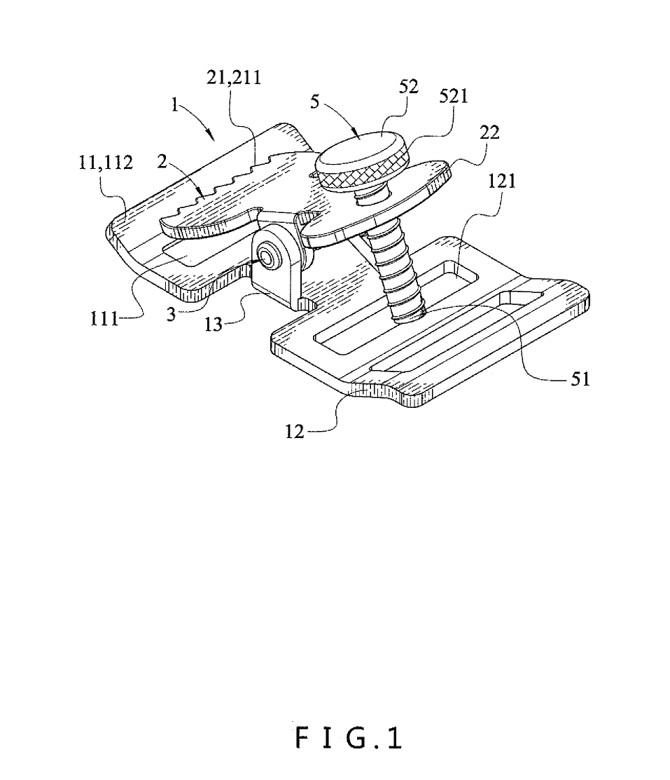

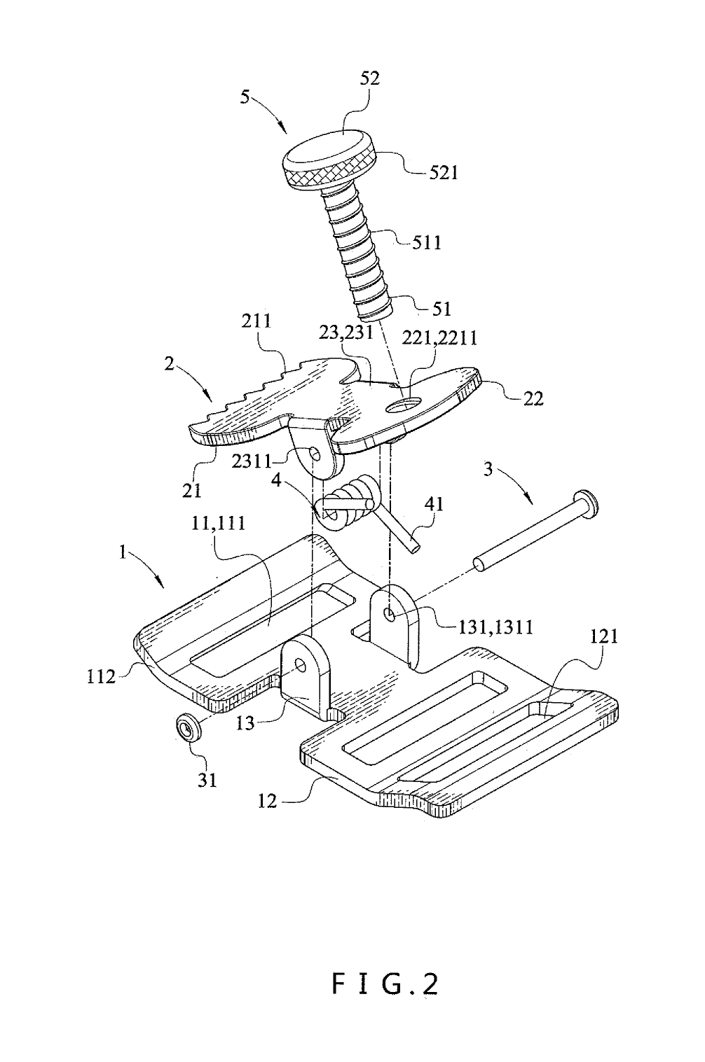

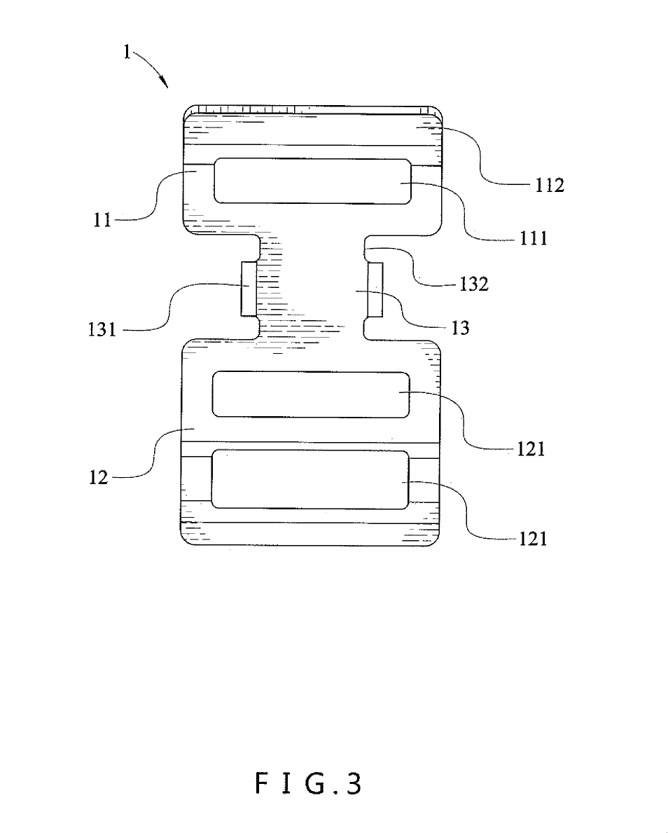

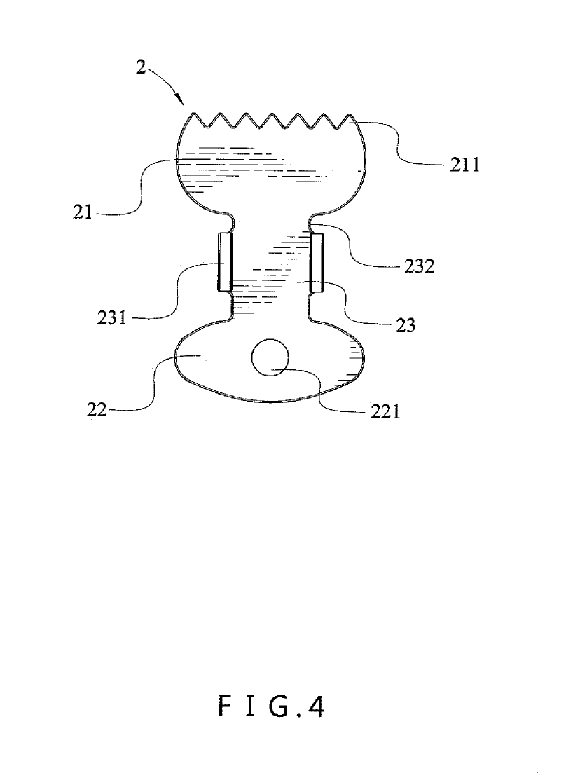

Referring to the drawings and initially to FIGS. 1-5, a compression clip structure in accordance with the preferred embodiment of the present invention comprises a first clip 1, a second clip 2, a shaft 3, an elastic member 4 and a fastener 5.

The first clip 1 has a first clamping portion 11, a mounting portion 12 and a first pivot portion 13. The first clamping portion 11 of the first clip 1 is provided with an opening 111 and a clamping zone 112. The mounting portion 12 of the first clip 1 is provided with at least one through hole 121. The first pivot portion 13 of the first clip 1 is located between the first clamping portion 11 and the mounting portion 12 and has two first ears 131 formed on two sides thereof. Each of the two first ears 131 of the first clip 1 is provided with a first pivot hole 1311.

The second clip 2 has a second clamping portion 21, a push portion 22 and a second pivot portion 23. The second clamping portion 21 of the second clip 2 is provided with a clamping end 211. The push portion 22 of the second clip 2 is provided with a fitting hole 221. The second pivot portion 23 of the second clip 2 is located between the second clamping portion 21 and the push portion 22 and has two second ears 231 formed on two sides thereof. Each of the two second ears 231 of the second clip 2 is provided with a second pivot hole 2311.

The shaft 3 extends through the first pivot hole 1311 of each of the two first ears 131 and the second pivot hole 2311 of each of the two second ears 231, so that the first clip 1 and the second clip 2 are connected pivotally by the shaft 3.

The elastic member 4 is mounted on the shaft 3 and provided with two resting portions 41 resting on the first clip 1 and the second clip 2, so that the clamping zone 112 of the first clip 1 and the clamping end 211 of the second clip 2 are directed toward each other, and the first clip 1 and the second clip 2 contain an elastic clamping force.

The fastener 5 extends through and is movable in the fitting hole 221 of the second clip 2. The fastener 5 is provided with a pressing portion 51 and a control portion 52. The control portion 52 of the fastener 5 controls movement of the pressing portion 51, so that the pressing portion 51 of the fastener 5 is located at a predetermined position.

In the preferred embodiment of the present invention, the first pivot portion 13 of the first clip 1 has two ends each formed with two first grooves 132 connected with the first clamping portion 11 and the mounting portion 12 respectively. Each of the two first ears 131 of the first clip 1 is bent upward along the two first grooves 132 of each of the two ends of the first pivot portion 13. In addition, the second pivot portion 23 of the second clip 2 has two ends each formed with two second grooves 232 connected with the second clamping portion 21 and the push portion 22 respectively. Each of the second ears 231 of the second clip 2 is bent downward along the two second grooves 232 of each of the two ends of the second pivot portion 23.

In the preferred embodiment of the present invention, the clamping end 211 of the second clip 2 has a serrated shape to increase the friction.

In the preferred embodiment of the present invention, a retainer 31 is mounted on the shaft 3 and abuts one of the two first ears 131 to retain the shaft 3. Thus, the first clip 1 and the second clip 2 are pivoted about the shaft 3.

In the preferred embodiment of the present invention, the control portion 52 of the fastener 5 has a periphery provided with a knurled area 521.

In the preferred embodiment of the present invention, the elastic member 4 is a torsion spring.

In the preferred embodiment of the present invention, the fitting hole 221 of the second clip 2 is provided with an internal thread 2211, and the pressing portion 51 of the fastener 5 is provided with an external thread 511 screwed into the internal thread 2211 of the second clip 2.

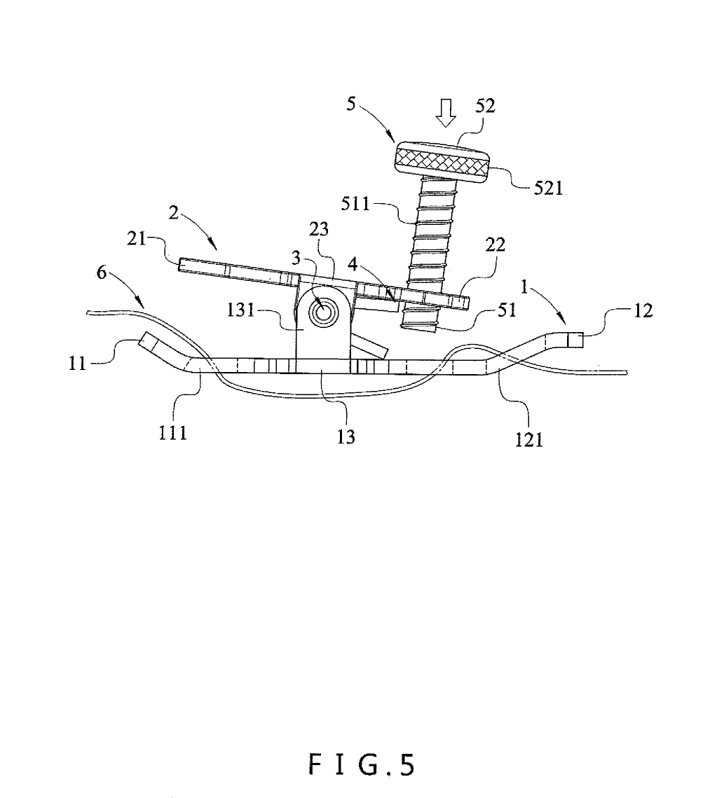

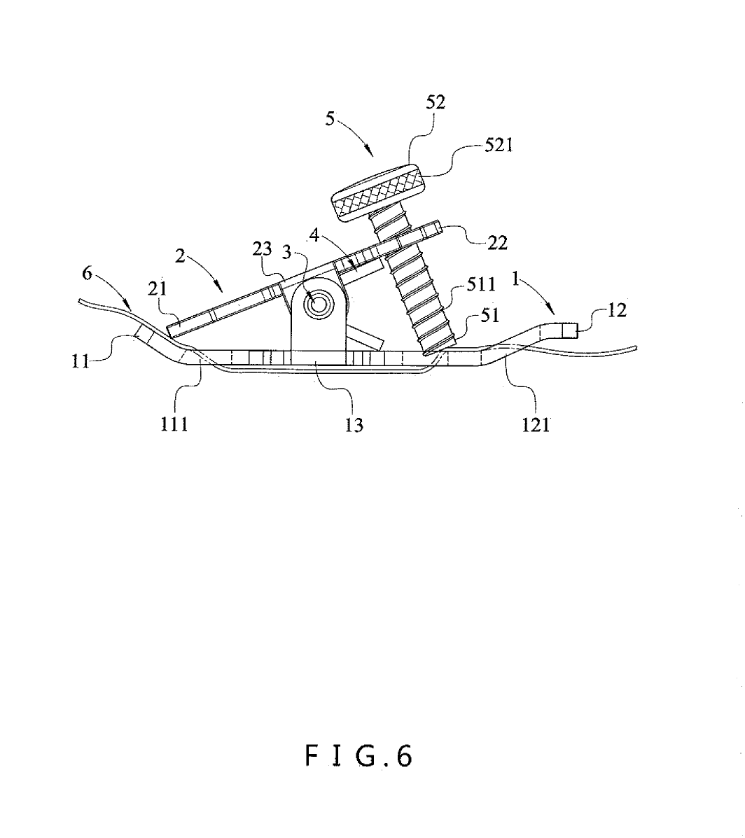

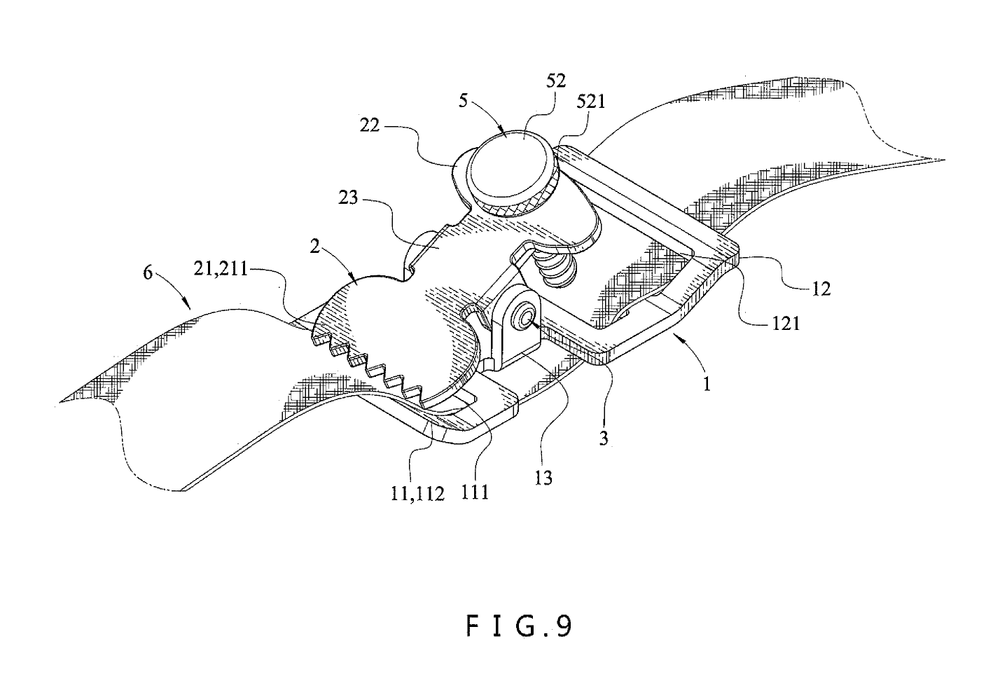

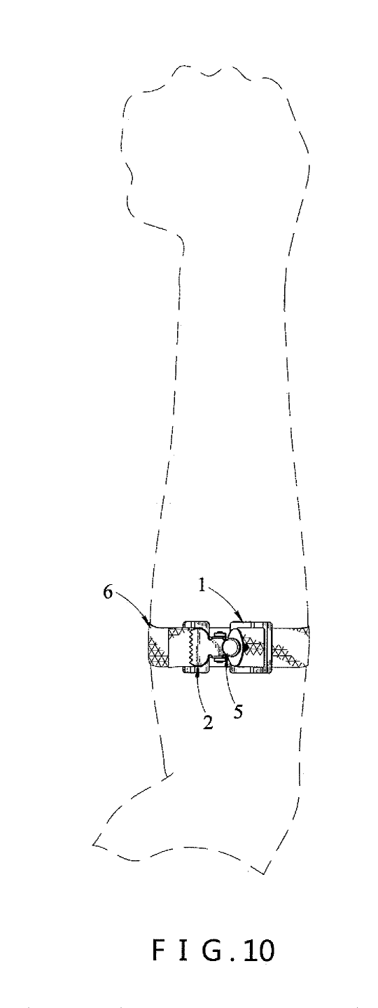

In operation, referring to FIGS. 5-10 with reference to FIGS. 1-4, a compression bandage 6 extends through the opening 111 and the at least one through hole 121 of the first clip 1. The clamping zone 112 of the first clip 1 and the clamping end 211 of the second clip 2 are used to clamp and secure the compression bandage 6. When the compression bandage 6 extends through the first clip 1, the pressing portion 51 of the fastener 5 directly or indirectly rests on the second clip 2, so that the first clip 1, the second clip 2 and the compression bandage 6 are disposed at a secured combination state. The external thread 511 of the fastener 5 is screwed into the internal thread 2211 of the second clip 2, so that when the control portion 52 of the fastener 5 is rotated, the fastener 5 is moved relative to the second clip 2 and the first clip 1 to adjust the predetermined position of the pressing portion 51 of the fastener 5. Thus, when the pressing portion 51 of the fastener 5 is located at the position as shown in FIG. 5, the first clip 1 is pivoted relative to the second clip 2, and when the pressing portion 51 of the fastener 5 is located at the position as shown in FIG. 6, the first clip 1 and the second clip 2 are secured by the fastener 5.

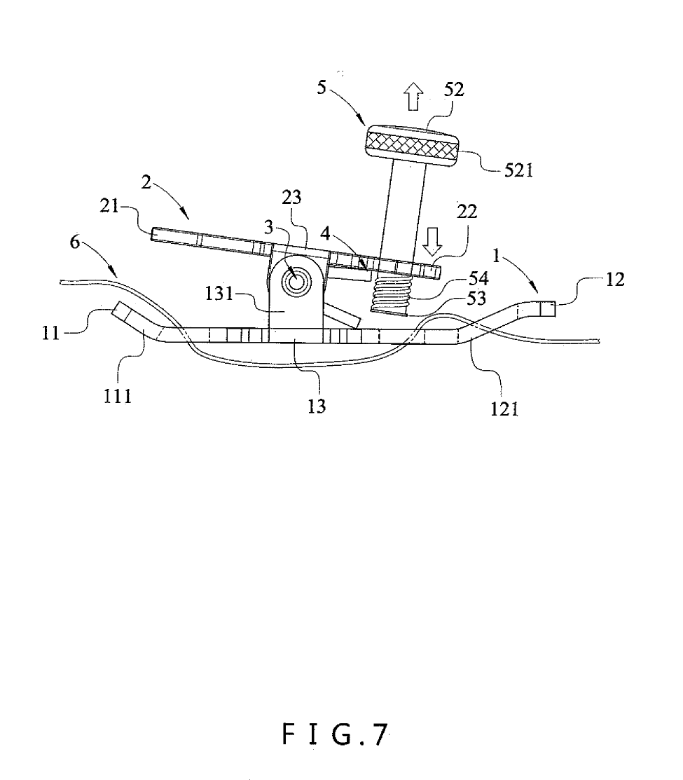

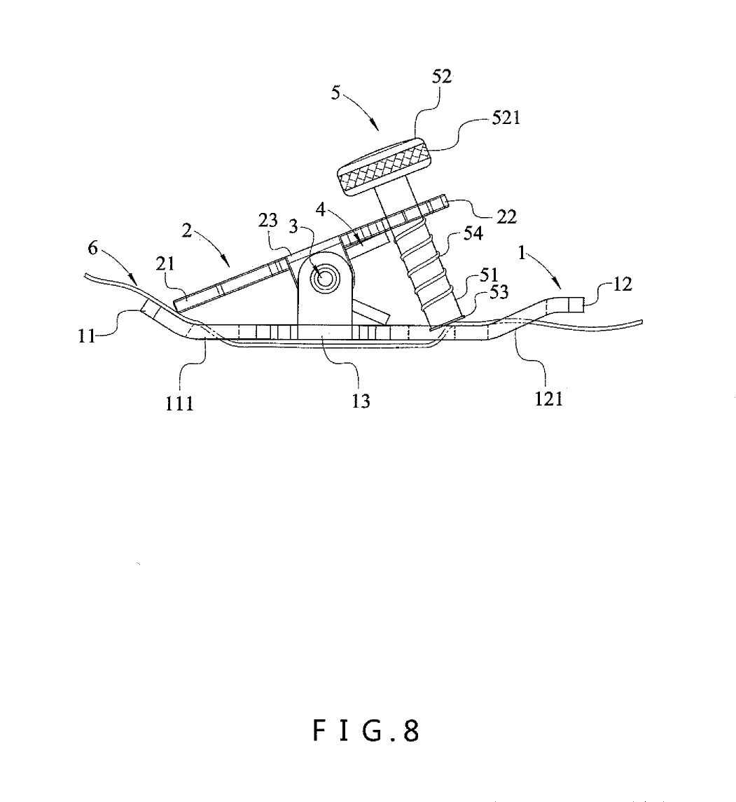

As shown in FIGS. 7 and 8, the pressing portion 51 of the fastener 5 has a lower end provided with an annular flange 53, and a spring 54 is mounted on the pressing portion 51 of the fastener 5 and is biased between the annular flange 53 of the fastener 5 and the second clip 2. The spring 54 has a lower end pressing the annular flange 53 of the fastener 5 and an upper end pressing the second clip 2. Thus, the pressing portion 51 of the fastener 5 is pressed downward by the elastic force of the spring 54.

The compression clip structure further comprises a movable securing mechanism mounted on the fastener 5 and the fitting hole 221 of the second clip 2. After the fastener 5 is pulled upward and the compression clip structure is mounted on the compression bandage 6, the compression bandage 6 is bound on a human body and located at a position between the heart and the wounded part. Thus, the first clip 1, the second clip 2, the shaft 3, the elastic member 4 and the fastener 5 are combined to secure the compression bandage 6, so as to stop the bleeding.

Accordingly, the compression clip structure is used to clamp the compression bandage 6, and the fastener 5 is used to secure the compression clip structure and the compression bandage 6, so as to stop the bleeding stably.

Although the invention has been explained in relation to its preferred embodiment(s) as mentioned above, it is to be understood that many other possible modifications and variations can be made without departing from the scope of the present invention. It is, therefore, contemplated that the appended claim or claims will cover such modifications and variations that fall within the scope of the invention.

1. A compression clip structure comprising:
a first clip, a second clip, a shaft, an elastic member and a fastener;
wherein:
the first clip has a first clamping portion, a mounting portion and a first pivot portion;
the first clamping portion of the first clip is provided with an opening and a clamping zone;
the mounting portion of the first clip is provided with at least one through hole;
the first pivot portion of the first clip is located between the first clamping portion and the mounting portion and has two first ears formed on two sides thereof;
each of the two first ears of the first clip is provided with a first pivot hole;
the second clip has a second clamping portion, a push portion and a second pivot portion;
the second clamping portion of the second clip is provided with a clamping end;
the push portion of the second clip is provided with a fitting hole;
the second pivot portion of the second clip is located between the second clamping portion and the push portion and has two second ears formed on two sides thereof;
each of the two second ears of the second clip is provided with a second pivot hole;
the shaft extends through the first pivot hole of each of the two first ears and the second pivot hole of each of the two second ears, so that the first clip and the second clip are connected pivotally by the shaft;
the elastic member is mounted on the shaft and provided with two resting portions resting on the first clip and the second clip, so that the clamping zone of the first clip and the clamping end of the second clip are directed toward each other, and the first clip and the second clip contain an elastic clamping force;
the fastener extends through and is movable in the fitting hole of the second clip;
the fastener is provided with a pressing portion and a control portion; and
the control portion of the fastener controls movement of the pressing portion, so that the pressing portion of the fastener is located at a predetermined position.

2. The compression clip structure of claim 1, wherein:
the first pivot portion of the first clip has two ends each formed with two first grooves connected with the first clamping portion and the mounting portion respectively; and
each of the two first ears of the first clip is bent upward along the two first grooves of each of the two ends of the first pivot portion.

3. The compression clip structure of claim 2, wherein:
the second pivot portion of the second clip has two ends each formed with two second grooves connected with the second clamping portion and the push portion respectively; and
each of the second ears of the second clip is bent downward along the two second grooves of each of the two ends of the second pivot portion.

4. The compression clip structure of claim 1, wherein the clamping end of the second clip has a serrated shape.

5. The compression clip structure of claim 1, wherein a compression bandage extends through the opening and the at least one through hole of the first clip.

6. The compression clip structure of claim 5, wherein the clamping zone of the first clip and the clamping end of the second clip clamp and secure the compression bandage.

7. The compression clip structure of claim 6, wherein when the compression bandage extends through the first clip, the pressing portion of the fastener directly or indirectly rests on the second clip, so that the first clip, the second clip and the compression bandage are disposed at a secured combination state.

8. The compression clip structure of claim 1, wherein a retainer is mounted on the shaft and abuts one of the two first ears to retain the shaft, so that the first clip and the second clip are pivoted about the shaft.

9. The compression clip structure of claim 1, wherein the control portion of the fastener has a periphery provided with a knurled area.

10. The compression clip structure of claim 1, wherein the elastic member is a torsion spring.
